(19) United States
(12) Patent Application Publication
(10) Pub. No.: US 2020/0108014 A1
(43) Pub. Date: Apr. 9, 2020

(54) CANNABIDIOL FORMULATION AND METHODS OF MAKING AND USING

(71) Applicant: Little River Band of Ottawa Indians, Manistee, MI (US)

(72) Inventors: Matthew Zoeller, Chicago, IL (US); Garrick Opie, Suttons Bay, MI (US)

(73) Assignee: Little River Band of Ottawa Indians, Manistee, MI (US)

(21) Appl. No.: 16/389,436

(22) Filed: Apr. 19, 2019

Related U.S. Application Data
(60) Provisional application No. 62/742,656, filed on Oct. 8, 2018.

(51) Int. Cl. A61K 9/107; A61K 9/00; A61K 47/10; A61K 47/24; A61K 47/44; A61K 31/05

(52) U.S. Cl. CPC A61K 9/1075; A61K 9/0014; A61K 47/10; B82Y 5/00; A61K 47/44; A61K 31/05; A61K 47/24

(57) ABSTRACT

A formulation for topical application to humans includes an emulsion of micelles in a lipid phase dispersed in an aqueous phase. Each micelle has cannabidiol (CBD) oil and at least one surfactant, and each micelle has a diameter less than about 100 nanometers, and preferably less than 10 nanometers. The CBD oil is present in a range of 3 mg to 100 mg per fluid ounce (30 ml) of emulsion. The aqueous phase includes a mixture of water and polyols or alcohols. The formulation is made by mixing the surfactant and cannabidiol (CBD) oil in a disperse phase, mixing an aqueous phase comprising the water and polyols or alcohols, heating the disperse phase and aqueous phase to about 80 degrees centigrade, slowly transferring the disperse phase into the aqueous phase with moderate sheer and agitation to form the emulsion; and cooling the emulsion to less than 50 degrees centigrade while maintaining a slow to moderate mixing speed.

### CROSS REFERENCE TO RELATED APPLICATIONS

This application claims the benefit of U.S. Application Ser. No. 62/742,636 filed Oct. 8, 2018, the entirety of which is incorporated herein.

### BACKGROUND

There are at least 80 known phytocannabinoid components isolated from the Cannabis sativa plant. Many are under study for their physiological activity when ingested and applied topically to human skin. Cannabidiol, known chemically as 2-[3-methyl-6-(1-methylethenyl)-2-cyclohexen-1-yl]-5-pentyl-1,3-benzenediol (CBD), and its related alkyl, ethoxy, and hydroxyalkyl substituted variants have known therapeutic uses. As continued studies reveal new therapeutic uses for CBD, there is greater need for accurate and reliable delivery of CBD and related compounds to enhance the desired treatment benefits.

### BRIEF SUMMARY

Aspects of the present disclosure relate to a cannabidiol (CBD) formulation, and methods of making and using. According to one aspect of the present disclosure, a CBD formulation includes disperse phase micelle vesicles bearing cannabidiol (CBD).

### DETAILED DESCRIPTION

Aspects of the present disclosure relate to delivery of the lipid fraction, comprising miscible broad-spectrum cannabidiol (CBD) lipid using a dermatologically safe delivery product, more specifically, a nanoscale emulsion having very small micellar size. This internally dispersed lipid phase has a particle size smaller than 100 nanometers (nm), optionally less than 10 nm, in a nano-emulsion having stable rheology. At this very small scale, these micelles/vesicles are light refracting and make the emulsion appear translucent to clear, usually characterized as a ringing gel. Standard oil-in-water emulsions have a micelle disperse phase size greater than 1 micrometer (1000 nm). Aspects of the present disclosure relate to the use of a nanoscale lipid carrier that is designed to transport CBD subcutaneously through the interstitial spaces of the dermis, channeling through the barrier layers to deliver the benefit of CBD to the subcutaneous regions.

CBD is dispersed into the internal or disperse phase of the nano-emulsion. The preferred amount is a dosage effective to deliver benefit to a human. A useful range of CBD application topically to humans will be 3 mg (0.003 g) to 100 mg (0.100 g) per fluid ounce (30 ml). Emulsification occurs when the surface tension of the continuous (or aqueous) phase is lowered, by inclusion of polyols in the aqueous phase and inclusion of the surfactants carrying the disperse (lipid/oil) phase. The disperse phase is the lipid phase, comprising oils and primary surfactants. The continuous phase is the aqueous phase. The free energy of the system is lowered sufficiently to form micelles (lipid/surfactant vesicles.) The dispersion may be facilitated by adequate miscibility of the CBD with organic solvents. Suitable solvent carriers can be selected from a range of organic solvents known to be compatible with human skin and safety. The choice of solvent carrier for the CBD disperse phase can be derived from the class of organic compounds used in the formulation of cosmetic health and beauty products which are applied directly to human skin. According to one aspect, the solvent carrier is selected from solvents that are relatively non-polar in chemical character and are able to maintain cohesive disperse phase stability, optionally in combination with selecting solvents that are safe for use with human skin.

Non-limiting examples of suitable solvent carriers may be from the family of homologs of alkyl, oleic, and alkyl substituted aromatic esters. These may include alkyl esters of benzoic acid, naphthenic acid, phthalic acid and similar alkyl esters of the homolog series C8 to C22 fatty acid monoesters and alcohols. These carriers may also be derived from saturated petroleum derivatives such as mineral oil, petrolatum and microcrystalline waxes. These carriers may also be derived from lower carbon chain linear alkyl esters such as amyl laurate or moieties from the homolog monoester series C4 up to C40 alkyl chains. These lipid solvents may also be derived from homologs in the mono-unsaturated and poly-unsaturated oleic acid esters and alcohol series of similar carbon chain length, C4 to C40.

According to an aspect of the present disclosure, the disperse phase is less than 10 nm, and thus may be classified as nanotechnology related. Optionally, a micellar size of 100 nanometers will be enough to generate the keratin penetration properties for a dosage form of CBD suitable for application with humans according to one aspect of the present disclosure.

The rheology of the CBD formulations of the present disclosure may be stabilized by a group of amphiphile surfactants. According to one aspect, no single surfactant working alone will suffice to create the desired nano-emulsion qualities. Suitable amphiphile surfactants are generally based on organic compounds having capacity to greatly reduce surface tension at the oil/water interface. These may be selected from classes of anionic, nonionic, and ethoxylated compounds where the alkyl (lipid) moiety of the surfactant is given enhanced hydrophilic character by adduct of ethylene oxide polymerization.

In one example, the series of mono-unsaturated oleyl and alkyl ethers are employed as co-surfactants (i.e. Oleth-5, Ceteareth-25, Laureth-7). These may also include ether adducts of dodecyl (C12), lauryl (C14), stearyl (C18), oleic (C18:1) and other homologs of saturated and unsaturated alkyl and oleic esters. Such surface-active agents may also derive from ethoxylated adducts of lanolin and of beeswax (i.e. PEG-25 hydrogenated lanolin, PEG-7 Beeswax.)

According to one aspect, suitable surfactants include the alkyl and oleic homolog series of phosphate esters, which are anionic in character (i.e. Oleth-10 Phosphate). The lipophilic character of these phosphate esters can be modified in situ by pH modification using an appropriate alkali. These phospholipids may also be derived from the homologous series of amino acid phosphor-esters, including phosphatidylcholine, phosphatidylserine, and other homologs. These phosphor-esters are strong emulsifying agents and are compatible with human physiology, as they are present in the cell membrane. The alkali adjusting agent may be from primary, secondary and tertiary alkyl amines, such as ethanolamine, morpholine, or amino functional nitroparaffins. Other suitable alkali can be selected from the range of PEGylated amines such as PEG-8 cocamine.

According to an aspect of the present disclosure, the disperse phase may be a combination of (a) linear and branched paraffinics, (b) alkyl, aromatic, or oleic esters, and (c) CBD. This disperse phase can be subsequently emulsified at 80° C. in a continuous phase including mainly water and polyols. Dispersion occurs when both phases are heated and homogeneous at 80° C., then the disperse phase (oil phase) is slowly transferred into the continuous phase (aqueous phase) with moderate sheer and agitation in a steam jacketed sanitary stainless steel (schedule 304 or 316) vessel. This mixture is then cooled to a temperature range acceptable for testing and packaging, about 40-50° C. Mixing speed is slow to moderate in the cooling sequence. The emulsion can be referred to as a nano-emulsion because it includes a lipid disperse phase emulsified within a continuous phase comprising a mixture of water, diols, polyols, linear alcohols, or non-cyclical sugar polyols. The resulting emulsion, having very low surface tension between phases, forms dispersed lipid vesicles having diameters of less than about 50 nm, optionally less than about 10 nm. The emulsification process of the present disclosure can be abetted by use of amphiphilic surfactants, such as those described above. According to an aspect of the present disclosure, the selected polyols and alcohols are also safe for human topical contact. These may include, but are not limited to, glycerin, propylene glycol, butylene glycol, hexylene glycol, sorbitol, ethoxydiglycol, dipropylene glycol and other polyols such as xylitol. According to one aspect, more than one polyol is incorporated into the continuous phase to produce a stable nano-emulsion. Only one polyol is needed in most cases, but more than one has benefits. Selection of polyols may depend on desired entropic stability, appearance, tactile feel on skin and other properties. Generally, the diols (e.g. propylene glycol, butylene glycol, pentylene glycol) suit stability best; while glycerin is most commonly found in commercial hair-grooming products using micro-emulsions.

In summary, the emulsion includes (a) a solvent carrier (e.g., a variety of esters, or mineral oil, petrolatum and microcrystalline waxes), stabilized by (b) surfactants (e.g., from ethers, esters, or adducts of lanolin and of beeswax, and including (c) water, and (d) diols, polyols, linear alcohols, or non-cyclical sugar polyols. The solvent carrier can range from 5.0%-20.0% of the emulsion by weight. The surfactants can range from 5.0%-25.0% of the emulsion by weight. The water, preferably deionized, can range from 35.0%-75.0% of the emulsion by weight. And the polyols can range from 5.0%-20.0% of the emulsion by weight.

In this disclosure we consider “interstitial spaces” as the vertical spaces present in the keratin layers of hair, skin and nails. We consider “strata gaps” as the horizontal spaces between keratin layers. According to an aspect of the present disclosure, disperse phase micelle vesicles, bearing CBD, are configured to permeate the barrier function of the dermal layer due at least in part to their small dimension (diameters of less than about 50 nm, optionally less than about 10 nm) relative to the dimensions of the keratin layer interstitial spaces, which are in the range of 40-250 nm. This permeation can be advanced by osmotic pressure. The continuous phase of the nano-emulsion, consisting mostly of water, swells the dermal keratin as it hydrates it. Thus, the nano-emulsion employs the dual function of swelling skin strata via hydration while delivering CBD subcutaneously in a nanoscale delivery mode into the relatively wide spaces obtained.

Dermal keratin strata have variable gap widths and depths; this affects both the epidermis and subcutaneous layers. These dermal keratin strata gaps can vary between 100 nm and 2000 nm. The formulations of the present disclosure provide a lipid vesicle carrying a liquid payload (i.e., CBD) which can permeate these strata more effectively. According to one aspect, the vesicles are configured to have diameters less than about 50 nm, optionally less than 10 nm to facilitate permeation of the formulation. The manifold homeopathic benefits of CBD can be delivered according to the formulations of the present disclosure to the subcutaneous layer, but not the subdermal layer, where the health applications for human skin will be more pervasively applied.

To the extent not already described, the different features and structures of the various embodiments of the present disclosure may be used in combination with each other as desired. For example, one or more of the features illustrated and/or described with respect to one aspect can be used with or combined with one or more features illustrated and/or described with respect to the other aspects described herein. That one feature may not be illustrated in all of the embodiments is not meant to be construed that it cannot be, but is done for brevity of description. Thus, the various features of the different embodiments may be mixed and matched as desired to form new embodiments, whether or not the new embodiments are expressly described.

While aspects of the present disclosure have been specifically described in connection with certain specific embodiments thereof, it is to be understood that this is by way of illustration and not of limitation. Reasonable variation and modification are possible within the scope of the forgoing disclosure and drawings without departing from the spirit of the present disclosure which is defined in the appended claims.

What is claimed is:

1. A formulation for topical application to humans comprising:
an emulsion of micelles in a lipid phase dispersed in an aqueous phase,
each micelle having cannabidiol (CBD) oil and at least one surfactant, and
each micelle having a diameter less than about 100 nanometers.

2. The formulation of claim 1 wherein each micelle has diameter of less than about 10 nanometers.

3. The formulation of claim 1 further comprising at least one lipid solvent selected from a group including homologs of alkyl, oleic, and alkyl substituted aromatic esters, esters of benzoic acid, naphthenic acid, phthalic acid and similar alkyl esters of the homolog series C8 to C22 fatty acid monoesters and alcohols, mineral oil, petrolatum and microcrystalline waxes, amyl laurate or moieties from the homolog monoester series C4 up to C40 alkyl chains, and homologs in the mono-unsaturated and poly-unsaturated oleic acid esters and alcohol series of similar carbon chain length, C4 to C40.

4. The formulation of claim 1 wherein the at least one surfactant is selected from a group including alkyl and oleic homolog series of phosphate esters, and homologous series of amino acid phosphor-esters, including phosphatidylcholine, phosphatidylserine, and other homologs, and adducts of lanolin and of beeswax.

5. The formulation of claim 4 wherein the at least one surfactant is pH modified by an alkali selected from a group including primary, secondary and tertiary alkyl amines, such as ethanolamine, morpholine, or amino functional nitroparaffins, and a range of PEGylated amines such as PEG-8 cocamine.

6. The formulation of claim 1 wherein the aqueous phase includes a mixture of water and polyols or alcohols.

7. The formulation of claim 6 wherein the polyols or alcohols include at least one of diols, polyols, linear alcohols, or non-cyclical sugar polyols, glycerin, propylene glycol, butylene glycol, pentylene glycol, hexylene glycol, sorbitol, ethoxydiglycol, dipropylene glycol and xylitol.

8. The formulation of claim 1 wherein the CBD oil is present in a range of 3 mg to 100 mg per fluid ounce (30 ml) of emulsion.

9. A method of making a formulation for topical application to humans, the method comprising:
mixing at least one surfactant and cannabidiol (CBD) oil in a disperse phase;
mixing an aqueous phase comprising water and at least one polyol or alcohol;
heating the disperse phase and aqueous phase to about 80 degrees centigrade;
slowly transferring the disperse phase into the aqueous phase with moderate sheer and agitation in a steam jacketed sanitary vessel to maintain the temperature of the contents at about 80 degrees centigrade to form an emulsion; and
cooling the emulsion to less than 50 degrees centigrade while maintaining a slow to moderate mixing speed.

10. The method of claim 9 wherein the at least one surfactant is selected from a group including alkyl and oleic homolog series of phosphate esters, and homologous series of amino acid phosphor-esters, including phosphatidylcholine, phosphatidylserine, and other homologs, and adducts of lanolin and of beeswax.

11. The method of claim 9 wherein the CBD oil is present in a range of 3 mg to 100 mg per fluid ounce (30 ml) of emulsion.

12. The method of claim 9 further comprising modifying the pH of the at least one surfactant by adding an alkali.

13. The method of claim 12 wherein the alkali selected from a group including primary, secondary and tertiary alkyl amines, such as ethanolamine, morpholine, or amino functional nitroparaffins, and a range of PEGylated amines such as PEG-8 cocamine.

14. The method of claim 9 wherein the at least one polyol or alcohol includes at least one of diols, polyols, linear alcohols, or non-cyclical sugar polyols, glycerin, propylene glycol, butylene glycol, pentylene glycol, hexylene glycol, sorbitol, ethoxydiglycol, dipropylene glycol and xylitol.

15. A method of dosing cannabidiol (CBD) oil in a human, the method comprising:
applying topically to human skin a formulation of an emulsion of micelles in a lipid phase dispersed in an aqueous phase, wherein each micelle has CBD oil and at least one surfactant and each micelle has a diameter less than about 100 nanometers, and wherein the amount of CBD oils is in a range of 3 mg to 100 mg per fluid ounce (30 ml) of emulsion.

16. The method of claim 15 wherein the formulation includes at least one lipid solvent selected from a group including homologs of alkyl, oleic, and alkyl substituted aromatic esters, esters of benzoic acid, naphthenic acid, phthalic acid and similar alkyl esters of the homolog series C8 to C22 fatty acid monoesters and alcohols, mineral oil, petrolatum and microcrystalline waxes, amyl laurate or moieties from the homolog monoester series C4 up to C40 alkyl chains, and homologs in the mono-unsaturated and poly-unsaturated oleic acid esters and alcohol series of similar carbon chain length, C4 to C40.

17. The method of claim 15 wherein the at least one surfactant is selected from a group including alkyl and oleic homolog series of phosphate esters, and homologous series of amino acid phosphor-esters, including phosphatidylcholine, phosphatidylserine, and other homologs, and adducts of lanolin and of beeswax.

18. The method of claim 15 wherein the at least one surfactant is pH modified by an alkali selected from a group including primary, secondary and tertiary alkyl amines, such as ethanolamine, morpholine, or amino functional nitroparaffins, and a range of PEGylated amines such as PEG-8 cocamine.

19. The method of claim 15 wherein the aqueous phase includes a mixture of water and polyols or alcohols.

20. The method of claim 19 wherein the polyols or alcohols include at least one of diols, polyols, linear alcohols, or non-cyclical sugar polyols, glycerin, propylene glycol, butylene glycol, pentylene glycol, hexylene glycol, sorbitol, ethoxydiglycol, dipropylene glycol and xylitol.
